(19) United States
(12) Patent Application Publication
(10) Pub. No.: US 2026/0258454 A1
(43) Pub. Date: Sep. 3, 2026

(54) BIOCATALYTIC SYNTHESIS OF (1S,3S)-3-HYDROXYCYCLOHEXANE-1-CARBOXYLIC ACID COMPOUNDS

(71) Applicant: Bristol-Myers Squibb Company, Princeton, NJ (US)

(72) Inventors: Shane McKenna, Pensby (GB); Yichen Tan, East Brunswick, NJ (US); Michael J. Smith, Somerset, NJ (US); Richard J. Fox, Yardley, NJ (US); David Thomas George, Ewing, NJ (US); Candice Lee Joe, Metuchen, NJ (US)

(21) Appl. No.: 18/846,692

(22) PCT Filed: Mar. 14, 2023
(86) PCT No.: PCT/US2023/064277

§ 371 Date: Sep. 13, 2024

Related U.S. Application Data
(60) Provisional application No. 63/269,370, filed on Mar. 15, 2022.

(51) Int. Cl. C12P 7/42; B01J 31/24; C07F 1/04

(52) U.S. Cl. CPC C12P 7/42; B01J 31/2409; C07F 1/04; B01J 2231/643; B01J 2531/004; B01J 2531/824

(57) ABSTRACT

Improved methods and intermediates thereof for preparing (1S,3S)-3-hydroxycyclohexane-1-carboxylic acid are described. These compounds are useful as intermediates for making carbamoyloxymethyl triazole cyclohexyl acid LPA antagonists.

### CROSS-REFERENCE TO RELATED APPLICATION

This application claims the priority benefit of U.S. Provisional Application No. 63/269,370, filed Mar. 15, 2022; the content of which is herein incorporated by reference in its entirety.

### TECHNICAL FIELD

The present invention relates to improved methods for preparing (1S,3S)-3-hydroxycyclohexane-1-carboxylic acid and intermediates thereof.

### BACKGROUND

Carbamoyloxymethyl triazole cyclohexyl acid LPA (especially LPA1) antagonists that are useful for the treatment of fibrosis have been described. See, e.g., WO2017/223016 (US 2017/0360759). However, further improved methods of making intermediate (1S,3S)-3-hydroxycyclohexane-1-carboxylic acid, which provide practical, large-scale synthesis, and improved production quality, efficiency and safety, are needed.

### SUMMARY

The present invention provides improved methods for making (1S,3S)-3-hydroxycyclohexane-1-carboxylic acid. Also described is a significantly optimized overall process, including one-pot enzymatic cascade that produces the target compound with improved quality and efficiency.

### DETAILED DESCRIPTION

The features and advantages of the invention may be more readily understood by those of ordinary skill in the art upon reading the following detailed description. It is to be appreciated that certain features of the invention that are, for clarity reasons, described above and below in the context of separate embodiments, may also be combined to form a single embodiment. Conversely, various features of the invention that are, for brevity reasons, described in the context of a single embodiment, may also be combined so as to form sub-combinations thereof.

The skilled artisan will recognize that some chemical structures described herein may be represented on paper by one or more other resonance forms; or may exist in one or more other tautomeric forms, even when kinetically, the artisan recognizes that such tautomeric forms represent only a very small portion of a sample of such compound(s). Such compounds are clearly contemplated within the scope of this disclosure, though such resonance forms or tautomers are not explicitly represented herein.

In a 1st aspect, the invention provides a method of making a compound of Formula (I) (deoxy-functionalization/carbonylation):

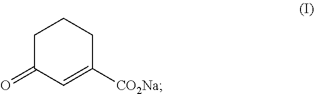

comprising (1) contacting a compound of Formula (II):

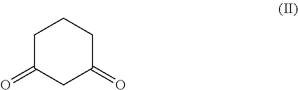

with a halogenating reagent or a sulfonylating reagent in a polar aprotic solvent or solvent mixture, with or without an inorganic or organic base;
for a time and at a temperature sufficient for deoxy-functionalization to produce a compound of Formula (III):

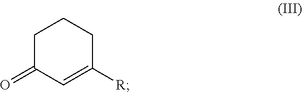

wherein R is Cl, Br, I, OMs, OTs, or OTf;
(2) with or without isolating the compound of Formula (III), contacting the compound of Formula (III) with a palladium catalyst, with or without a phosphine ligand, and an inorganic or organic base, in water and a polar aprotic solvent; purged with carbon monoxide;
for a time, at a temperature and pressure sufficient for carbonylation to produce the compound of Formula (I).

In a 2nd aspect, the invention provides a method of making a compound of Formula (I) (deoxy-functionalization/carbonylation):

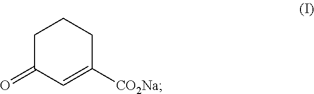

comprising (1) contacting a compound of Formula (II):

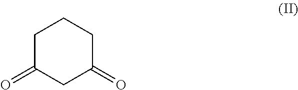

with a halogenating reagent selected from oxalyl iodide, oxalyl bromide, and oxalyl chloride or a sulfonylating reagent selected from p-toluenesulfonyl chloride, methanesulfonyl chloride, trifluoromethanesulfonic anhydride, N-phenyl-bis(trifluoromethanesulfonimide) and N-(5-chloropyridin-2-yl)-N-(methanesulfonyl)methanesulfonamide in a polar aprotic solvent selected from EtOAc, 2-MeTHF, CH₃CN, DMAc, DMF, THF, Toluene and a mixture thereof, with or without an inorganic or organic base selected from K₂CO₃, N,N-diisopropylethylamine, triethylamine, t-butyltetramethyl guanidine, tetramethyl guanidine, KHCO₃, DBU, Na₂CO₃, Na₂CO₃*10H₂O and KOAc;
for a time and at a temperature sufficient for deoxy-functionalization to produce a compound of Formula (III):

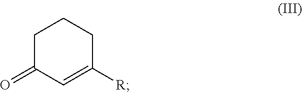

wherein R is Cl, Br, I, OMs, OTs, or OTf;
(2) with or without isolating the compound of Formula (III), contacting the compound of Formula (III) with a palladium catalyst selected from Pd(A-taPhos)₂Cl₂, Pd(DPEPhos)Cl₂, Pd(PPh₃)₂Cl₂, Pd(DCEPhos)Cl₂, Pd(OAc)₂, PdCl₂(CH₃CN)₂ and Pd(DPPP)Cl₂;
with or without a phosphine ligand selected from 1,3-bis(dicyclohexylphosphino)propane, 1,3-bis(diphenylphosphino)propane, rac-BINAP, Xantphos, Josiphos SL-J001-1, Josiphos SL-J009-1-G3 palladacycle, and BIPHEP; and
an inorganic or organic base selected from K₂CO₃, N,N-diisopropylethylamine, triethylamine, t-butyltetramethyl guanidine, tetramethyl guanidine, KHCO₃, DBU, Na₂CO₃, Na₂CO₃*10H₂O and KOAc;
in water and a polar aprotic solvent selected from EtOAc, 2-MeTHF, CH₃CN, DMAc, DMF, THF, Toluene and a mixture thereof;
   purged with carbon monoxide;
for a time, at a temperature and pressure sufficient for carbonylation to produce the compound of Formula (I).

In a 3rd aspect, the invention provides a method of making a compound of Formula (I) (deoxy-chlorination/carbonylation):

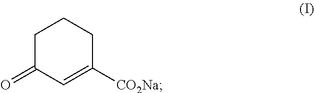

comprising (1) contacting a compound of Formula (II):

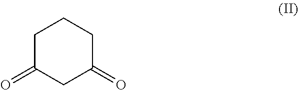

with acid chloride in a polar aprotic solvent or solvent mixture selected from 2-MeTHF and DMF;
for a time and at a temperature sufficient for deoxy-chlorination to produce a compound of Formula (IIIa):

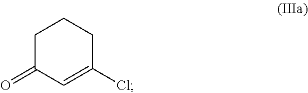

(2) with or without isolating the compound of Formula (IIIa), contacting the compound of Formula (IIIa) with Pd(A-taPhos)₂Cl₂, Pd(PPh₃)₂Cl₂, Pd(DCEPhos)Cl₂ or PdCl₂(CH₃CN)₂ with 1,3-bis(dicyclohexylphosphino)propane, and an organic or inorganic base selected from DIPEA, K₂CO₃, Na₂CO₃ and Na₂CO₃*10H₂O; in water and a polar aprotic solvent selected from 2-MeTHF, CH₃CN, Toluene, and a mixture thereof; purged with carbon monoxide;
for a time, at a temperature and pressure sufficient for carbonylation to produce the compound of Formula (I).

In a 4th aspect, the invention provides a method of making a compound of Formula (I):

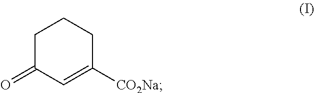

comprising (1) contacting a compound of Formula (II):

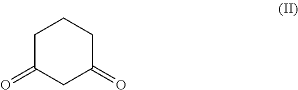

with oxalyl chloride in a mixture of 2-MeTHF and DMF;
for 1 to 3 hours and at 0 to 50° C. to produce a compound of Formula (IIIa):

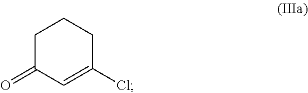

(2) with or without isolating the compound of Formula (IIIa), contacting the compound of Formula (IIIa) with Pd(A-taPhos)₂Cl₂ and Na₂CO₃, in water and 2-MeTHF; purged with carbon monoxide;
for 16 to 20 hours, at 20-50° C. and 30 to 35 psi to produce the compound of Formula (I).

In a 5th aspect, the invention provides a method of making a compound of Formula (IV):

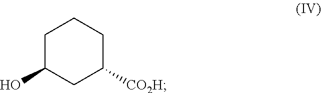

comprising contacting a compound of Formula (I):

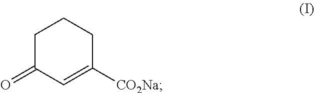

with an enone reductase enzyme (ERED) and a ketoreductase enzyme (KRED), in the presence of an aqueous polar protic solvent, NADPH and a co-substrate, with or without an organic cosolvent;
for a time and at a temperature sufficient to produce the compound of Formula (IV).

In a 6th aspect, the invention provides a method of making a compound of Formula (IV):

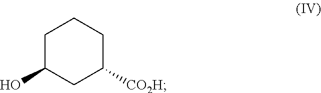

comprising contacting a compound of Formula (I):

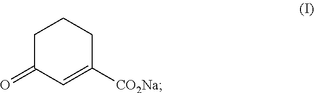

with an ERED selected from ERED-309 and ERED-310, and a KRED selected from KRED-456, KRED-457 and KRED-P2-B07; in the presence of an aqueous buffer selected from phosphate, TRIS, HEPES, ACES, BES, MOPS, and Tricene; GDH, NADPH and glucose with or without an organic cosolvent such as DMSO, IPA, dioxane or acetone;
for a time and at a temperature sufficient to produce the compound of Formula (IV).

In a 6a aspect, the invention provides a method of making a compound of Formula (IV):

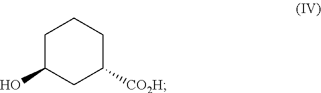

comprising contacting a compound of Formula (I):

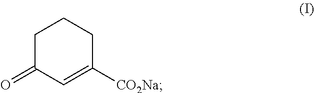

with an ERED selected from ERED-309 and ERED-310, and a KRED selected from KRED-456, KRED-457 and KRED-P2-B07, in the presence of an aqueous phosphate buffer, GDH, NADPH and glucose with or without an organic cosolvent such as DMSO, IPA, dioxane or acetone;
for a time and at a temperature sufficient to produce the compound of Formula (IV).

In a 7th aspect, the invention provides a method of making a compound of Formula (IV):

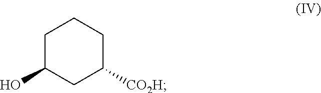

comprising contacting a compound of Formula (I):

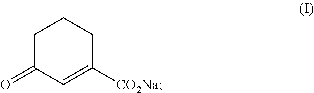

with ERED-310, and KRED 457, in the presence of an aqueous buffer selected from phosphate, TRIS, HEPES, ACES, BES, MOPS, and Tricene; GDH, NADPH and glucose with or without an organic cosolvent such as DMSO, IPA, dioxane or acetone;
for at least 16 hours and at 25 to 35° C. sufficient to produce the compound of Formula (IV).

In a 7a aspect, the invention provides a method of making a compound of Formula (IV):

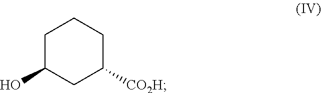

comprising contacting a compound of Formula (I):

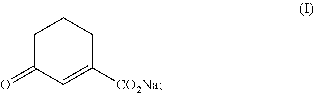

with ERED-310, and KRED 457, in the presence of an aqueous phosphate buffer, GDH, NADPH and glucose with or without an organic cosolvent such as DMSO, IPA, dioxane or acetone;
for at least 16 hours and at 25 to 35° C. sufficient to produce the compound of Formula (IV).

In an 8th aspect, the invention provides a method of making a compound of Formula (IV), comprising steps (1) and (2) from any of the 2nd to 4th aspects; then the step from any of the 5th to 7th aspects; wherein all formulae and variables are as defined as in the 2nd to 7th aspects.

In a 9th aspect, the invention provides a method of making a compound of Formula (IV), comprising steps (1) and (2) of the 3rd or 4th aspect; then the step of the 6th or 7th aspect; wherein all formulae and variables are as defined as in the 3rd, 4th, 6th, and 7th aspects.

In a 10th aspect, the invention provides a method of making a compound of Formula (IV), comprising steps (1) and (2) of the 4th aspect; then the step of the 7th aspect; wherein all formulae and variables are as defined as in the 4th and 7th aspects.

### Definitions

The presence of reaction impurities and/or processing impurities may be determined by analytical techniques known in the art, such as, for example, chromatography, nuclear magnetic resonance spectroscopy, mass spectrometry, and/or infrared spectroscopy.

Other embodiments include those described in the Detailed Description and/or in the claims.

To facilitate understanding of the disclosure set forth herein, a number of additional terms are defined below. Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs.

The term “halo” or “halogen” refers to fluoro (F), chloro (Cl), bromo (Br), or iodo (I).

The term “cycloalkyl” as used herein includes saturated cyclic hydrocarbon groups having 3 to 10 carbons, preferably 3 to 8 carbons, and more preferably 3 to 6 carbons, wherein the cycloalkyl group may be optionally substituted. Preferred cycloalkyl groups include, without limitation, cyclopropyl, cyclobutyl, cyclopentyl, cyclopentenyl, cyclohexyl, cyclohexenyl, cycloheptyl, and cyclooctyl. The term “cycloalkylene” as used herein refers to divalent cycloalkyl.

The term “Brønsted acid” refers to a proton (H⁺) donor.

The term “Lewis acid” refers to a chemical species that can accept an electron pair from an electron donor compound.

The term “Brønsted base” refers to a proton (H⁺) acceptor.

The term “Lewis base” refers to a chemical species that can donate an electron pair to an electron acceptor compound.

The term “transition metal catalyst” refers to a coordination complex that has any of various metallic elements such as palladium and nickel that have valence electrons in two shells instead of only one and, when added to a chemical reaction, increases the rate of reaction. Examples of palladium catalyst are, but not limited to, Pd(A-taPhos)₂Cl₂, Pd(DPEPhos)Cl₂, Pd(PPh₃)₂Cl₂, Pd(DCEPhos)Cl₂ or Pd(OAc)₂, PdCl₂(CH₃CN)₂ or Pd(DPPP)Cl₂.

The term “phosphine ligand” refers to a three valent phosphorous compound that can bond to a metal atom. Examples of phosphine ligand are, but not limited to, 1,3-bis(dicyclohexylphosphino)propane, 1,3-bis(diphenylphosphino)propane, rac-BINAP, Xantphos, Josiphos SL-J001-1, Josiphos SL-J009-1-G3 palladacycle, and BIPHEP.

A protic solvent refers to a solvent that has a hydrogen atom bound to an oxygen (as in a hydroxyl group) or a nitrogen (as in an amine group).

An aprotic solvent refers to a solvent that is not a hydrogen bond donor.

A polar solvent refers to a solvent with large dipole moments or partial charges; they contain bonds between atoms with very different electronegativities, such as oxygen and hydrogen.

Solvent mixture refers to a combination of two or more solvents.

The term “halogenating reagent” refers to a reagent that can introduce a halogen atom into a molecule.

The term “sulfonylating reagent” refers to a reagent that can introduce a sulfonate ester moiety into a molecule. Examples of sulfonate ester moiety are, but not limited to, mesylate (OMs), tosylate (OTs) and trifluoromethanesulfonate (triflate or OTf).

As used herein, “enone reductase,” “ene reductase,” and “ERED” are used interchangeably herein to refer to a polypeptide having a capability of reducing an α,β unsaturated compound to the corresponding saturated compound. More specifically, enone reductases are capable of reducing α, β unsaturated ketones, aldehydes, nitriles, olefins, and esters. For example, ERED-309 and ERED-310 can be obtained from Codexis, Inc.

As used herein, “ketoreductase” and “KRED” are used herein to refer to a polypeptide of the class (EC1.1.1.184), useful for the synthesis of optically active alcohols from the corresponding prochiral ketone substrate and by stereoselective reduction of corresponding racemic aldehyde substrates. KREDs typically convert ketone and aldehyde substrates to the corresponding alcohol product, but may also catalyze the reverse reaction, oxidation of an alcohol substrate to the corresponding ketone/aldehyde product. For example, KRED-456, KRED-457 and KRED-P2-B07 can be obtained from Codexis, Inc.

In addition, atoms making up the compounds of the present embodiments are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

### EXAMPLES

The following Examples are put forth so as to provide those of ordinary skill in the art with a complete disclosure and description of how to make and use the present invention, and are not intended to limit the scope of what the inventors regard as their invention, nor are they intended to represent that the experiments below were performed or that they are all of the experiments that may be performed. It is to be understood that exemplary descriptions written in the present tense were not necessarily performed, but rather that the descriptions can be performed to generate data and the like of a nature described therein. Efforts have been made to ensure accuracy with respect to numbers used (e.g., amounts, temperature, etc.), but some experimental errors and deviations should be accounted for.

The starting materials used in the synthetic sequence of the invention are known, made by known methods, or are commercially available. The skilled artisan will also recognize that conditions and reagents described herein that can be interchanged with alternative art-recognized equivalents. For example, in one reactions, hydrochloric acid can be interchanged with other acids, such as hydrobromic acid, sulfuric acid, etc.

The skilled artisan will recognize a variety of analytical methods that can be used to characterize the compounds described herein, including, for example, ¹H nuclear magnetic resonance spectroscopy (NMR), heteronuclear NMR, mass spectrometry (MS), liquid chromatography (LC), and infrared (IR) spectroscopy. The foregoing list is a subset of characterization methods available to a skilled artisan and is not intended to be limiting.

To further illustrate the foregoing, the following non-limiting, exemplary synthetic schemes are included. Variations of these examples within the scope of the claims are within the purview of one skilled in the art and are considered to fall within the scope of the invention as described, and claimed herein. The reader will recognize that the skilled artisan, provided with the present disclosure, and skill in the art is able to prepare and use the invention without exhaustive examples.

The following abbreviations have the indicated meanings:
ACES=N-(2-Acetarnido)-2-aminoethanesulfonic acid
aq=aqueous
BES=N,N- bis-(2-Hydroxyethyl)-2-aminoethanesulfonic Acid
BIPHEP=2,2′-Bis(diphenylphosphino)biphenyl
CH3CN=acetonitrile
CO=carbon monoxide
d=doublet
D₂O=deuterium oxide
DBU=1,8-diazabicyclo[5.4.0]undec-7-ene
DIPEA=N,N-diisopropylethylamine
DMAc=dimethylacetamide
DMF=dimethylformamide
DMSO=dimethylsulfoxide
equiv=equivalent(s)
ERED=enone reductase or ene reductase
ESI=electrospray ionization
EtOAc=ethyl acetate
g=gram(S)
GDH=Glucose dehydrogenase
h=hour(s)
HEPES=(N-2-hydroxyethylpiperazine-N-2-ethane sulfonic acid)
HCl=hydrogen chloride (usually as a solution)
H2O=water
HPLC=High Pressure Liquid Chromatography
IPA=isopropyl alcohol
Josiphos SL-J001-1=(2R)-1-[(1R)-1-(Dicyclohexylphosphino)ethyl]-2-(diphenylphosphino)ferrocene
Josiphos SL-J009-1-G3 palladacycle={(R)-1-[(Sp)-2-(Dicyclohexylphosphino)ferrocenyl]ethyldi-tert-butylphosphine}[2-(2′- amino-1,1′-biphenyl)]palladium(II) methanesulfonate
K₂CO₃=potassium carbonate
KHCO₃=potassium bicarbonate
KH₂PO₄=potassium phosphate monobasic
KF=Karl Fischer
KOAc=potassium acetate
kg=kilogram(s)
KRED=ketoreductase
L=liter(s)
LCAP=Liquid Chromatography Area Percent
m=multiplet
M=molar
MOPS=3-(N-morpholino)propanesulfonic acid
mg=milligram(s)
MeOH or CH₃OH=methanol
D4-MeOH=deuterated methanol
MeTHF or 2-MeTHF=2-methyltetrahydrofuran
MHz=megahertz
min=minute(s)
mL=milliliter(s)
mmol=millimole(s)
MS=Mass Spec
MSA=methane sulfonic acid
N₂=dinitrogen
NaCl=sodium Chloride
Na₂CO₃=sodium carbonate
Na₂CO₃*H₂O=sodium carbonate decahydrate
NADPH=nicotiiwamide adenine dinucleotide phosphate hydrogen
NaOH=sodium hydroxide
NaOMe=sodium methoxide
Na₂SO₄=sodium sulfate
NH₄Cl=ammonium Chloride
NMT=not more than
Pd(A-taPhos)₂Cl₂=Bis(di-tert-butyl(4-dimethylaminophenyl) phosphine)dichloropalladium(II)
PdCl₂(CH₃CN)₂=Bis(acetonitrile)dichloropalladium(II)
Pd(DCEPhos)Cl₂=Dichloro[bis(dicyclohexylphosphinophenyl) ether]palladium(II)
Pd(DPEPhos)Cl₂=Dichloro{bis[2-(diphenylphosphino)phenyl]ether}palladium(II)
Pd(DPPP)Cl₂=[1,1′-Bis(diphenylphosphino)ferrocene]dichloropalladium(II)
Pd(OAC)₂=palladium(II) acetate
Pd(PPh₃)₂Cl₂=Bis(triphenylphosphine)palladium(II) dichloride
ppm=parts per million
q=quartet
rac-BINAP=racemic (2,2′-bis(diphenylphosphino)-1,1′-binaphthyl)
RT=retention time
s=singlet
t=triplet
td=triplet of doublets
THF=tetrahydrofuran
Tol=toluene
Tricene=N-[Tris-(hydroxymethy)methyl]glycine
TRIS=tromethamine
° C.=degrees Celsius
UPLC/MS=Ultra Pressure Liquid chromatography Mass Spectrometer
vol=volumes
wt=weight
Xantphos=[5-(diphenylphosphino)-9,9-dimethyl-9H-xanthen-4-yl](diphenyl)-phosphine

A number of embodiments of the invention have been described. Nevertheless, it will be understood that various modifications may be made without departing from the spirit and scope of the invention. Accordingly, other embodiments are within the scope of the following claims.

### Experimental Methods

### Deoxy-chlorination/Carbonylation

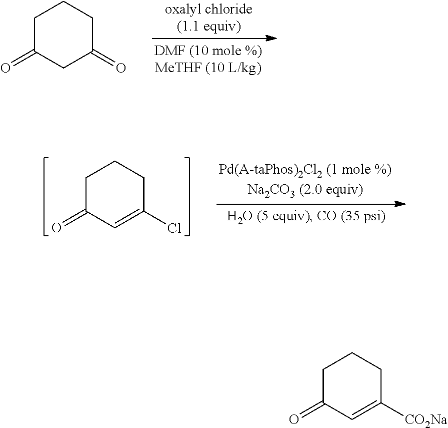

### Example 1. Preparation of Sodium 3-oxocyclohex-1-ene-1-carboxylate

### Step 1a:

A 250 mL jacketed Chemglass reactor was charged with dione (10.0 g, 1.0 equiv.), MeTHF (150.0 mL, 15.0 L/kg), and DMF (0.69 mL, 0.1 equiv.). The jacket temperature was set to 5.0° C. cooling the reaction mass to an internal temperature of <7.5° C. Once cooled oxalyl chloride (8.41 mL, 1.1 equiv.) was added via syringe pump over 30 minutes (CAUTION: Gas evolution! CAUTION: Exotherm observed. Internal temperature did not exceed 10.0° C.). After the addition the jacket temperature was set to 20.0° C. warming the reaction mass to 20.0° C. After 90 minutes the reaction mass was sampled for conversion and indicated complete consumption of the starting dione (NMT 1.0 RAP starting dione). The jacket temperature was set to 5.0° C. cooling the reaction mass to an internal temperature of <7.5° C. Once cooled 5 wt % NH₄Cl_{aq}. (30 mL, 3.0 L/kg) was added in a period not less than 30 minutes. (CAUTION: Gas evolution! CAUTION: Exotherm). After the addition the jacket temperature was set to 20.0° C. warming the reaction mass to 20.0° C. After 10 minutes the agitation was shut off and the phases were allowed to settle. The phases were split sending the aq. layer to waste. Agitation was resumed and the stream was treated with 15 wt % NaCl_{aq}. (20 mL, 2.0 L·kg). After 10 minutes the agitation was shut off and the phases were allowed to settle. The phases were split sending the aq. layer to waste. The pressure of the reactor was decreased to 150 torr and the jacket temperature was set to 55.0° C. Once the reaction mass was concentrated to 4.0 L/kg the jacket temperature was set to 20.0° C. and the pressure increased to 760 torr. After cooling to an internal temperature of 20.0° C. the process stream was polish filtered.

### Step 1 b:

An inerted 300 mL HEL reactor was charged with sodium carbonate (12.3 g, 2.0 equiv.), PdCl₂(A-taphos)₂ (651.0 mg, 0.01 equiv.), MeTHF (100.0 mL, 10.0 L/kg), the vinyl chloride reaction solution (vide supra), and water (8.0 mL, 5.0 equiv.). The reactor was sealed, pressurized with 20 psi N₂ and vented to ambient pressure (repeat purge procedure 3×). The vessel was pressurized with 30 psi CO and vented to ambient pressure (repeat purge procedure 3×). The reactor was pressurized with 35 psi CO and heated the contents to 50° C. (Note: Ensure that agitation is high to suspend inorganic base and to ensure good gas-liquid mixing). After 16 hours, the vessel was cooled to 20° C. and vented to ambient pressure. The vessel was then pressurized with 20 psi N₂ and vented to ambient pressure (repeat purge procedure 3×). The reaction mass was sampled for conversion and indicated complete consumption of the vinyl chloride intermediate (NMT 1.0 RAP vinyl chloride remaining).

The reaction mass was quenched with water (50.0 mL, 5.0 L/kg) and acidified with 6 N HCl (25.4 mL, 2.0 equiv.) (NOTE: Reaction heterogeneous prior to acidification, stream becomes biphasic and homogeneous after acidification). The phases were split sending the aqueous layer to waste (NOTE: Sampling organic layer typically indicates >90% in-process yield. NOTE: KF typically 3.5 wt %. NOTE: pH 1.0-1.5). After splitting the organic layer is polished filtered into a clean reactor. The pressure of the reactor was decreased to 150 torr and the jacket temperature was set to 55.0° C. Once the reaction mass was concentrated to 5.0 L/kg the jacket temperature was set to 20.0° C. and the pressure increased to 760 torr. After cooling to an internal temperature of 20.0° C. the process stream was polish filtered into a clean reactor. The transfer line and filter were rinsed with MeTHF (30.0 mL, 3.0 L/kg) sending the rinse into the clean reactor containing the process stream (NOTE: KF typically 1.6 wt %). Charged a 25 wt % solution of NaOMe in MeOH (17.5 mL, 1.0 equiv.) dropwise over 90 minutes (NOTE: pH should not exceed 6.5). The resulting slurry was aged for 2 hours and subsequently filtered. The reactor and filter cake was washed with 4:1 v/v MeTHF:MeOH (30 mL, 3.0 L/kg). The filter cake was washed with MeTHF (30.0 mL, 3.0 L/kg). The wet cake was dried in an oven vacuum at 45.0° C. under a sweep of nitrogen for a period not less than 7 hours. The sodium carboxylate product was as an off-white solid (>75%).

¹H NMR (500 MHz, D₂O) δ 6.34 (1H, s), 2.54 (2H, td), 2.45 (2H, t), 2.02 (2H, q).

MS: Molecular Formula, Exact Mass Expected; Found —C₇H₈O₃ (free acid), Exact Mass Expected 140.0473: High-res mass spec data not yet collected.

HPLC method conditions: Column: Zorbox Eclipse Plus C8, 150×4.6 mm, 3.5 μm; Mobile phase A: 0.05% MSA in water:Acetonitrile 98:2+10 mM KH2PO4; Mobile phase B: 0.05% MSA in water:Acetonitrile 10:90; Temperature: 50° C.; Gradient: 0 min (2% B), 4.0 min (20% B), 8.0 min (30% B); 10.0 min (90% B); 12.0 min (90% B); 12.1 min (2% B), 17.0 min (2% B), Flow: 0.8 mL/min; 210 nm; HPLC RT 7.0 min.

### Alternative Experimental Procedure:

### Step 1a:

A glass-lined reactor was charged with MeTHF (10.0 L/kg), DMF (0.1 eq), and 1,3-cyclohexanedione (1.0 eq). The jacket temperature was set to maintain an internal temperature of 10-20° C. (target 15° C.). Oxalyl chloride (1.0 eq) was charged via metered addition (20-40 kg/h) (CAUTION: Gas evolution! CAUTION: Exotherm observed). After 1.0 h the reaction mass was sampled for conversion of the starting dione (NMT 0.5 RAP starting dione). If residual dione remains charge additional oxalyl chloride (0.1 eq.) and re-sample the reaction mass after aging an additional 1.0 h. Adjust the pH with DIPEA (~1.0 eq.) until pH 6.0-7.0. Water (1.5 L/kg) was charged and the resulting biphasic mixture was agitated for 0.5-1.0 h. After agitation was stopped, the phases were allowed to separate for NLT 1.0 h. The phases were split sending the aq. layer to waste. Agitation was resumed and the process stream was treated with 15 wt % NaCl_{aq}. (2.0 L/kg). The resulting biphasic mixture was agitated for 0.5-1.0 h. Agitation was stopped, and the phases were allowed to separate for NLT 1.0 h. The phases were split sending the aq. layer to waste.

### Step 1 b:

An autoclave reactor was charged with MeTHF (5.0 L/kg) and the process stream from Step 1a. The combined process stream was sampled for water content. If necessary, the KF of the process stream was adjusted to ensure the presence of ≥1.2 eq. of water. The process stream was treated with Na₂CO₃ (1.2 eq) and Pd(A-taPhos)₂Cl₂ (0.01 eq). The atmosphere of the autoclave was exchanged with nitrogen and carbon monoxide. The vessel was pressurized with carbon monoxide (0.2-0.3 MPa) and the reaction mass was warmed to 30-40° C. (target 35° C.). After 3.0 h the reaction mass was sampled for conversion of the starting vinyl chloride. Once s 80.0 RAP vinyl chloride remained then the reaction mass was warmed to 45-55° C. (target 50° C.) and aged for an additional 16.0 h. After aging, the process stream was sampled for conversion of starting vinyl chloride (NMT 1.0 RAP starting vinyl chloride). The reaction mass was cooled to 0-5° C. (target 2.5° C.). The process stream was quenched with water (10.0 L/kg) and the resulting biphasic mixture was agitated for 1.0-2.0 h. The biphasic mixture was filtered over a nutsche filter into a glass-lined reactor. The autoclave reactor and nutsche filter were rinsed with water (10.0 L/kg) sending the rinse to the same glass-lined reactor. The pH of the mixture was adjusted with 6 N HCl to 1.0-2.0 (target 1.5) and the biphasic mixture was agitated for NLT 0.5 h. After agitation was stopped, the phases were allowed to separate for NLT 1.0 h. The phases were split sending the aq. layer to waste. Agitation was resumed and the process stream was treated with 15 wt % NaCl_{aq}. (1.5 L/kg) The resulting biphasic mixture was agitated for 0.5-1.0 h. Agitation was stopped, and the phases were allowed to separate for NLT 1.0 h. The phases were split sending the aq. layer to waste. Agitation was resumed and the process stream was treated with 15 wt % NaCl_{aq}. (1.5 L/kg) The resulting biphasic mixture was agitated for 0.5-1.0 h. After agitation was stopped, the phases were allowed to separate for NLT 1.0 h. The phases were split sending the aq. layer to waste. The process stream was treated with active carbon (0.1 g/g) and agitated for NLT 1.0 h. The process stream was filtered, and the filter cake washed with MeTHF (3.0 L/kg). The pressure of the reactor was decreased, and the jacket temperature was set to 55.0° C. Once the reaction mass was concentrated to 4.0-5.0 L/kg the jacket temperature was set to −15.0-5.0° C. (target −10° C.). The stream was diluted with MeTHF (3.5 L/kg) and the KF of the stream was adjusted to 1.0-2.0 wt %. 20 wt % NaOMe in MeOH was charged in a metered fashion until the pH registered 6.0-7.0. The resulting slurry was agitated for an additional 2.0 h. After aging, the slurry was filtered and washed with 4:1 v/v MeTHF: MeOH (2.0 L/kg) and MeTHF (2.0 L/kg). The wet cake was dried in an oven vacuum at 45.0° C. for NLT 7.0 h. Isolated the sodium carboxylate product as an off-white solid (>75%).

### Enone Reductase/Ketoreductase Cascade

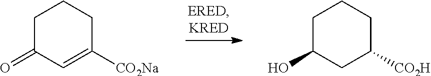

### Example 2: Bio-Catalytic Synthesis of (1S,3S)-3-hydroxycyclohexane-1-carboxylic Acid

To a 100 mL reactor was add ERED-310 (80 mg, 8 wt %), GDH-105 (2 mg, 0.002 wt %), NADP+(10 mg, 0.1 wt %) and KRED-457 (40 mg, 4 wt %). Then 0.25 M sodium phosphate buffer pH 7.0 (10 mL, 10 L/Kg) was added and the agitation set to 300 rpm. The reaction was allowed to age for 30 minutes to allow dissolution of the biocatalysts. The pH was then adjusted to pH 7.0 with 2.5 M aqueous NaOH, followed by adding D-Glucose (2.4 g, 2.16 equiv). At this point, temperature was set to 32.5° C. and reaction pH=7.0 was maintained using a pH stat. In a second vessel, sodium 3-oxocyclohex-1-ene-1-carboxylate was charged (1.0 g, limiting reagent), 0.25 M sodium phosphate buffer pH 7.0 (5 mL, 5 L/Kg) Glucose 0.6 g (0.54 equiv) and agitated for 10 minutes. The pH of this solution was then adjusted to pH 7.0 by the addition of 2.5 M NaOH. This solution was then charged to the biocatalyst solution over 6 hours via syringe pump. After 16 hours, the reaction was judged complete (>99 LCAP).

Upon completion of the reaction, cellulose (2.0 g, 200 wt %) was added to the reaction mixture, and 6N HCl (aq) was added to adjust the pH to 1-2. The resulting slurry was stirred at room temperature for a period not less than 1 hour before filtration. The filter cake was thoroughly washed by MeTHF (20 mL, 20 L/Kg) to minimize yield loss. The resulting biphasic filtrate was charged with solid Na₂SO₄ (3 g, 300 wt %), and stirred until all the Na₂SO₄ was dissolved. The phases were split sending the aqueous layer to waste. The organic layer is transferred into a clean vial. Concentrated the stream under 100 torr at 35.0° C. Once the reaction mixture was concentrated to 2.5 L/kg, MeTHF (5 mL, 5.0 L/Kg) was added, and the reaction mixture was concentrated again to 2.5 L/Kg. Repeat the process until the KF of the stream is NMT 0.5 wt %. The process stream was polish filtered into a clean vial. Charged a 1/3 Tol/heptane mixture (3 mL, 3 L/Kg) dropwise over 90 minutes. The resulting slurry was aged for 3 hours and additional 1/3 Tol/heptane mixture (7 mL, 7 L/Kg) was added slowly over 3 hours. The resulting slurry was aged for 4 hours at room temperature before cooled to 0° C. and aged for 3 hours. The slurry was then filtered. The vial and filter cake were washed with 1/3 Tol/heptane (2 mL, 2.0 L/kg). The wet cake was dried in an oven vacuum at 50.0° C. under a sweep of nitrogen for a period not less than 8 hours. The product (1S,3S)-3-hydroxycyclohexane-1-carboxylic acid was obtained as an off-white solid 70-80%).

¹H NMR (400 MHz, d₄-MeOH) δ 3.93-4.00 (m, 1H), 2.67-2.75 (m, 1H), 1.65-1.85 (m, 4H), 1.45-1.63 (m, 4H).

13C NMR: (100 MHz, d4-MeOH) δ 179.64, 66.88, 39.11, 36.51, 33.75, 29.27, 21.05.

MS: Molecular Formula, Exact Mass Expected; Found. 144.08 g/mol.

LC/MS (DCI) Calcd for [C₇H₁₂O₃+H]⁺=145.086, found 145.0865.

Analytical: Working concentration 5 mg/mL, Injection volume: 5 μL. Column: Ascentis Express C18, PCP, 4.6×150 mm, 2.7 μm, Flow: 0.8 mL/min Colum Temp 25° C., Mobile Phase A 0.1% Formic Acid in Water, Mobile Phase B 0.1% Formic Acid in MeCN, UV wavelength (210 nm), Gradient 98% A (0.0 min), 80% A (4 min), 50% A (8 min), 10% A (10 min), 98% A (10.1 min), 98% A (15 min) RT(UPLC): 5.0 min.

### Alternative Experimental Procedure:

To a 100 mL reactor was add ERED-310 (80 mg, 8 wt %), GDH-105 (2 mg, 0.2 wt %), NADP+ (10 mg, 1 wt %) and KRED-457 (30 mg, 3 wt %). Then 0.1 M Sodium Phosphate buffer pH 7.0 with 2 mM MgCl₂ (12 mL, 12 L/Kg) was added and the agitation set to 300 rpm. The reaction was allowed to age for 30 minutes to allow dissolution of the biocatalysts. The pH was then adjusted to pH 7.0 with 2.5M aqueous NaOH, followed by adding D-Glucose (2.4 g, 2.4×). At this point, set internal temperature to 30° C. and utilized a pH stat to maintain reaction pH=7.0.

In a second vessel, charged sodium 3-oxocyclohex-1-ene-1-carboxylate (1.0 g, limiting reagent), 0.1 M Sodium Phosphate buffer pH 7.0 with 2 mM MgCl₂ (3 mL, 3 L/Kg), Glucose 0.6 g (0.6 g/g), NADP+ (5 mg, 0.5 wt %) and agitated for 10 minutes. The pH of this solution was then adjusted to pH 7.0 by the addition of 2.5 M NaOH or 2.5M HCl. This solution was then charged to the biocatalyst solution over 8-10 hours via syringe pump. During the addition, utilize a pH stat to maintain reaction pH=7.0.

After 16 hours, the reaction was judged complete (>99% conversion of sodium 3-oxocyclohex-1-ene-1-carboxylate). Upon completion of the reaction, cellulose (1.0 g, 1×) was added to the reaction mixture, and the reaction mixture was heated to 50-60° C. and stirred for 1 h. The mixture was cooled to 15-30° C. and 6N HCl (ag) was added to adjust the pH to 1-2. The resulting slurry was stirred at room temperature for NLT 1 hour before filtration. The filter cake was thoroughly washed by MeTHF (20 mL, 20 L/Kg) to minimize yield loss. The resulting biphasic filtrate was charged with solid Na₂SO₄ (3 g, 3×) and stirred until all the Na₂SO₄ was dissolved. The phases were split sending the aqueous layer to waste. The organic layer is transferred into a clean vessel. The Stream was distilled under 100 torr at 35.0° C. Once the reaction mixture was concentrated to 5 L/kg, MeTHF (5 mL, 5.0 L/Kg) was added, and the reaction mixture was concentrated again to 5 L/Kg. Repeat the process until the KF of the stream is NMT 0.5 wt % (usually two put-and-take). The process stream was polish filtered into a clean Vessel. The reaction was then concentrated again to 2.5 L/Kg. To the concentrated solution, was charged Tol/heptane mixture (1/3, 3 mL, 3 L/Kg) dropwise over 90 minutes. The resulting slurry was aged for 3 hours and additional 1/3 Tol/heptane mixture (7 mL, 7 L/Kg) was added slowly over 3 hours. The resulting slurry was aged for 2 hours at room temperature before cooled to 0° C. and aged for 1 hours. The slurry was then filtered. The reactor and filter cake were washed with 1/3 Tol/heptane (2 mL, 2.0 L/kg). The wet cake was dried in an oven vacuum at 50° C. under a sweep of nitrogen for NLT 8 hours. Obtained the product (1S,3S)-3-hydroxycyclohexane-1-carboxylic acid as an off-white solid (70-80%).

It is to be appreciated that the Detailed Description section, and not the Summary and Abstract sections, is intended to be used to interpret the claims. The Summary and Abstract sections may set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

1. A method of making a compound of Formula (I): 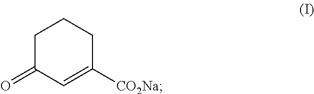
comprising (1) contacting a compound of Formula (II) thereof: 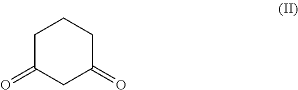
with a halogenating reagent or a sulfonylating reagent in a polar aprotic solvent or solvent mixture, with or without an inorganic or organic base;
for a time and at a temperature sufficient for deoxy-functionalization to produce a compound of Formula (III): 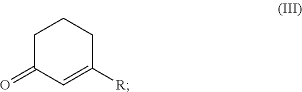
wherein R is Cl, Br, I, OMs, OTs, or OTf;
(2) with or without isolating the compound of Formula (III), contacting the compound of Formula (III) with a palladium catalyst, with or without a phosphine ligand, and an inorganic or organic base, in water and a polar aprotic solvent; purged with carbon monoxide;
for a time, at a temperature and pressure sufficient for carbonylation to produce the compound of Formula (I).

2. The method of making a compound of Formula (I) according to claim 1,
comprising (1) contacting a compound of Formula (II) with a halogenating reagent selected from oxalyl iodide, oxalyl bromide, and oxalyl chloride or a sulfonylating reagent selected from p-toluenesulfonyl chloride, methanesulfonyl chloride, trifluoromethanesulfonic anhydride, N-phenyl-bis(trifluoromethanesulfonimide) and N-(5-chloropyridin-2-yl)-N-(methanesulfonyl)methanesulfonamide in a polar aprotic solvent selected from EtOAc, 2-MeTHF, CH₃CN, DMAc, DMF, THF, Toluene and a mixture thereof, with or without an inorganic or organic base selected from K₂CO₃, N,N-diisopropylethylamine, triethylamine, t-butyltetramethyl guanidine, tetramethyl guanidine, KHCO₃, DBU, Na₂CO₃, Na₂CO₃*10H₂O and KOAc;
for a time and at a temperature sufficient for deoxy-functionalization to produce a compound of Formula (III), wherein R is Cl, Br, I, OMs, OTs, or OTf;
(2) with or without isolating the compound of Formula (III), contacting the compound of Formula (III) with a palladium catalyst selected from Pd(A-taPhos)₂Cl₂, Pd(DPEPhos)Cl₂, Pd(PPh₃)₂Cl₂, Pd(DCEPhos)Cl₂, Pd(OAc)₂, PdCl₂(CH₃CN)₂ and Pd(DPPP)Cl₂;
with or without a phosphine ligand selected from 1,3-bis(dicyclohexylphosphino)propane, 1,3-bis(diphenylphosphino)propane, rac-BINAP, Xantphos, Josiphos SL-J001-1, Josiphos SL-J009-1-G3 palladacycle, and BIPHEP; and
an inorganic or organic base selected from K₂CO₃, N,N-diisopropylethylamine, triethylamine, t-butyltetramethyl guanidine, tetramethyl guanidine, KHCO₃, DBU, Na₂CO₃, Na₂CO₃*10H₂O and KOAc;
in water and a polar aprotic solvent selected from EtOAc, 2-MeTHF, CH₃CN, DMAc, DMF, THF, Toluene and a mixture thereof;
purged with carbon monoxide;
for a time, at a temperature and pressure sufficient for carbonylation to produce the compound of Formula (I).

3. The method of making a compound of Formula (I) according to claim 2,
comprising (1) contacting a compound of Formula (II) with acid chloride in a polar aprotic solvent or solvent mixture selected from 2-MeTHF and DMF;
for a time and at a temperature sufficient for deoxy-chlorination to produce a compound of Formula (IIIa): 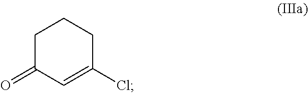
(2) with or without isolating the compound of Formula (IIIa), contacting the compound of Formula (IIIa) with Pd(A-taPhos)₂Cl₂, Pd(PPh₃)₂Cl₂, Pd(DCEPhos)Cl₂ or PdCl₂(CH₃CN)₂ with 1,3-bis(dicyclohexylphosphino)propane, and an organic or inorganic base selected from DIPEA, K₂CO₃, Na₂CO₃ and Na₂CO₃*10H₂O; in water and a polar aprotic solvent selected from 2-MeTHF, CH₃CN, Toluene, and a mixture thereof; purged with carbon monoxide;
for a time, at a temperature and pressure sufficient for carbonylation to produce the compound of Formula (I).

4. The method of making a compound of Formula (I) according to claim 3,
comprising (1) contacting a compound of Formula (II) with oxalyl chloride in a mixture of 2-MeTHF and DMF;
for 1 to 3 hours and at 0 to 50° C. to produce a compound of Formula (IIIa);
(2) with or without isolating the compound of Formula (Illa), contacting the compound of Formula (IIIa) with Pd(A-taPhos)₂Cl₂ and Na₂CO₃, in water and 2-MeTHF; purged with carbon monoxide;
for 16 to 20 hours, at 20-50° C. and 30 to 35 psi to produce the compound of Formula (I).

5. A method of making a compound of Formula (IV): 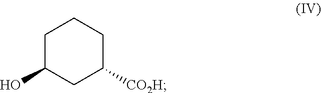
comprising contacting a compound of Formula (I) thereof: 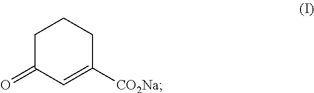
with an enone reductase enzyme (ERED) and a ketoreductase enzyme (KRED), in the presence of an aqueous polar protic solvent, NADPH and a co-substrate, with or without an organic cosolvent;
for a time and at a temperature sufficient to produce the compound of Formula (IV).

6. The method of making a compound of Formula (IV) according to claim 5,
comprising contacting a compound of Formula (I) with an ERED selected from ERED-309 and ERED-310, and a KRED selected from KRED-456, KRED-457 and KRED-P2-B07; in the presence of an aqueous buffer selected from phosphate, TRIS, HEPES, ACES, BES, MOPS, and Tricene; GDH, NADPH and glucose with or without an organic cosolvent such as DMSO, IPA, dioxane or acetone;
for a time and at a temperature sufficient to produce the compound of Formula (IV).

7. The method of making a compound of Formula (IV) according to claim 6,
comprising contacting a compound of Formula (I) with ERED-310, and KRED 457, in the presence of an aqueous buffer selected from phosphate, TRIS, HEPES, ACES, BES, MOPS, and Tricene; GDH, NADPH and glucose with or without an organic cosolvent such as DMSO, IPA, dioxane or acetone;
for at least 16 hours and at 25 to 35° C. sufficient to produce the compound of Formula (IV).

8-10. (canceled)

11. A method of making a compound of Formula (IV): 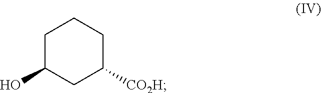
comprising (1) contacting a compound of Formula (II): 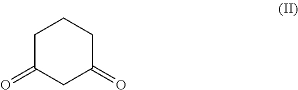
with oxalyl chloride in a mixture of 2-MeTHF and DMF;
for 1 to 3 hours and at 0 to 50° C. to produce a compound of Formula (IIIa): 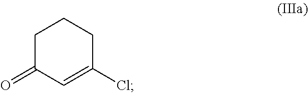
(2) with or without isolating the compound of Formula (IIIa), contacting the compound of Formula (IIIa) with Pd(A-taPhos)₂Cl₂ and Na₂CO₃, in water and 2-MeTHF; purged with carbon monoxide;
for 16 to 20 hours, at 20-50° C. and 30 to 35 psi to produce the compound of Formula (I): 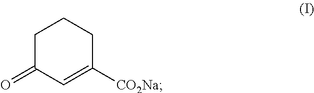
then (3) contacting a compound of Formula (I) with ERED-310, and KRED 457, in the presence of an aqueous buffer selected from phosphate, TRIS, HEPES, ACES, BES, MOPS, and Tricene; GDH, NADPH and glucose with or without an organic cosolvent such as DMSO, IPA, dioxane or acetone;
for at least 16 hours and at 25 to 35° C. sufficient to produce the compound of Formula (IV).
